SEQUENCE LISTING (11 sequences)

SEQ ID NO: 1 - DNA, 30 bp, Artificial Sequence
  Description of Artificial Sequence Primer for Stimulated PCR
gccgtcttccagttgctttatctgttcact

SEQ ID NO: 2 - DNA, 30 bp, Artificial Sequence
  Description of Artificial Sequence Primer for Stimulated PCR and Half-Nested PCR
cctgatggcaaatgccccaattgcaggtaa

SEQ ID NO: 3 - DNA, 33 bp, Artificial Sequence
  Description of Artificial Sequence Primer for Stimulated PCR
gtcaagtagcatctgtatcaggcaaagtcatag

SEQ ID NO: 4 - DNA, 30 bp, Artificial Sequence
  Description of Artificial Sequence Primer for Half-Nested PCR
tgttcacttgtgccctgactttcaactctg

SEQ ID NO: 5 - DNA, 24 bp, Artificial Sequence
  Description of Artificial Sequence Primer for Sequencing
tgccctgactttcaactctgtctc

SEQ ID NO: 6 - DNA, 20 bp, Artificial Sequence
  Description of Artificial Sequence Primer for Sequencing
agggtccccaggcctctgat

SEQ ID NO: 7 - DNA, 22 bp, Artificial Sequence
  Description of Artificial Sequence Primer for Sequencing
ggccactgacaaccacccttaa

SEQ ID NO: 8 - DNA, 20 bp, Artificial Sequence
  Description of Artificial Sequence Primer for Sequencing
aggtctccccaaggcgcact

SEQ ID NO: 9 - DNA, 20 bp, Artificial Sequence
  Description of Artificial Sequence Primer for Sequencing
ggggcacagcaggccagtgt

SEQ ID NO: 10 - DNA, 19 bp, Artificial Sequence
  Description of Artificial Sequence Primer for Sequencing
ggagagaccggcgcacaga

SEQ ID NO: 11 - DNA, 24 bp, Artificial Sequence
  Description of Artificial Sequence Primer for Sequencing
cggcattttgagtgttagactgga

(19) United States
(12) Patent Application Publication
(10) Pub. No.: US 2003/0049635 A1
(43) Pub. Date: Mar. 13, 2003

(54) Measurement of mutation load using the p53 gene in human cells from paraffin embedded tissues

(75) Inventors: Steven S. Sommer, Duarte, CA (US); Qiang Liu, Upland, CA (US); Ernst Heinmoller, Kessel (DE)

Correspondence Address: ROTHWELL, FIGG, ERNST & MANBECK, P.C., 1425 K STREET, N.W., SUITE 800, WASHINGTON, DC, 20005, US

(73) Assignee: City of Hope, Duarte, CA

(21) Appl. No.: 09/986,381

(22) Filed: Nov. 8, 2001

Related U.S. Application Data
(60) Provisional application No. 60/246,582, filed on Nov. 8, 2000.

(51) Int. Cl. C12Q 1/68; G01N 33/574; C12P 19/34

(52) U.S. Cl. 435/6; 435/7.23; 435/91.2

(57) ABSTRACT

A method for determining mutation load in a somatic cell is determined by mutation analysis of the p53 gene. The p53 gene has been found to be a useful indicator of predisposition to spontaneous mutations or prior carcinogen exposure. Cells that contain mutated p53 tend to accumulate the mutant protein. Thus, DNA from a cell identified by p53 accumulation is amplified and the amplification product further analyzed for mutations in the p53 gene.

This application claims priority from U.S. Provisional Application No. 60/246,582, filed Nov. 8, 2000.

### BACKGROUND OF THE INVENTION

This invention relates to a method for determining the extent and nature of mutations of somatic origin. The method is capable of utilizing as little DNA as that obtained from a single cell, and may reveal predisposition to elevated spontaneous mutations as well as prior carcinogen exposure. Thus the invention provides a useful tool for determining cancer risk or monitoring the effectiveness of cancer therapy.

Somatic mutations can compromise genome integrity. Mutation analysis of DNA from a single somatic cell or a small group of cells is a useful procedure, having a number of applications. For example, analysis of somatic mutations within single cells permits examination of “mutation load,” defined herein as the overall mutation frequency and alterations in mutation pattern and spectrum. These measurements in normal tissues can identify individuals with a high mutation load who are at an increased risk for cancer. Since mutation patterns can vary dramatically with the type of mutagenic insult or defective repair system, they can provide clues to identify mutation sources and/or mechanisms. In addition, mutation analysis of a single cell or small group of cells may be useful for identifying specific mutations that may be associated with a potentially pathological event or a general tool in molecular pathology.

It has become clear over the last decade that cancer is, in essence, a genetic disease resulting from accumulation of mutations in specific oncogenes and tumor suppressor genes, either through inherited or somatic origins. The p53 tumor suppressor gene is a critical regulator of tumorigenesis and is mutated in 50% of human cancers. Levine, A. J., “The p53 tumor-suppressor gene”, N. Engl. J. Med. 326:1350-1352 (1992); Hollstein, M., Sidransky, D., Vogelstein, B., Harris, C. C., “p53 mutations in human cancer”, Science 253:49-53 (1992). Moreover, individuals with Li-Fraumeni syndrome, who inherit a p53 mutant allele, develop multiple cancers at an early age. Malkin, D., et. al., “Germ line p53 mutations in a familial syndrome of breast cancer, sarcomas, and other neoplasms”, Science 250:1233-1238 (1990). Additionally, transgenic mice with a nullizygous or a mutant p53 gene develop normally but develop cancers within 3 to 6 months after birth. Donehower, L. A., et. al., Mice deficient for p53 are developmentally normal but susceptible to spontaneous tumours”, Nature 356:215-221 (1992). Furthermore, the p53 gene is associated with responses to various cancer treatments, including, but not limited to, radiation, cytotoxic drug treatment and gene therapy treatment. Lowe, S. W., et. al., “p53 status and the efficacy of cancer therapy in vivo”, Science 266:807-810 (1994).

The p53 gene is located in a 20 kb region of chromosome 17p13.1. The gene contains 11 exons which encode a nuclear phosphoprotein of 393 amino acids that is expressed in all cell types. The p53 protein can be divided into three major regions based on function. Prives, C., “How loops, beta sheets, and alpha helices help us to understand p53”, Cell 78:543-546 (1994). The acidic amino-terminal contains the transcriptional activation region and a binding site for the product of the mdm2 gene. The central region is necessary for sequence specific DNA binding and contains the binding sites for SV40 large T-antigen. The carboxyl-terminal region contains a domain necessary for p53 oligomerization, one primary and two secondary nuclear localization signal sequences and sequences mediating non-specific DNA binding.

Despite previous speculations that the p53 gene is “the guardian of the genome,” compelling data indicate that the frequency and pattern of single-base changes and microdeletions or insertions are unchanged in normal tissues from p53 nullizygous mice that develop cancer early. The background rate of mutation in p53 null cells is similar to that of wild type cells. Sands, A. T., et. al., “p53 deficiency does not affect the accumulation of point mutations in a transgene target”, Proc. Natl. Acad. Sci. U.S.A. 92:8517-8521 (1995); Nishino, H., et. al., “p53 wild-type and p53 nullizygous Big Blue transgenic mice have similar frequencies and patterns of observed mutation in liver, spleen and brain”, Oncogene 1:263-270 (1995).

The p53 protein is accumulated by missense mutations in exons 5 through 9, making possible immunohistochemical staining (IHCS) of mutant cells. Ninety-five percent of mutations in the p53 gene occur in exons 5 to 9. Soussi, T., et. al., “Structural aspects of the p53 protein in relation to gene evolution”, Oncogene 5:945-952 (1990). Proliferating cell nuclear antigen (PCNA; cytosolic ICHS) is essential in DNA replication and DNA repair. Nonmutated p53 is a regulator of the level of expression of multiple genes, including PCNA, mdm2 and vEGF. Mutated p53 may result in altered regulation of these proteins. For example, while wild-type p53 downregulates PCNA, mutated p53, when present in high concentration, can activate the PCNA promoter and can result in increased expression of PCNA. Jackson, P., et. al., “Transcriptional regulation of the PCNA promoter by p53”, Biochem. Biophys. Res. Commun. 203:133-140 (1994). However, tissues occasionally contain normal cells that exhibit accumulated p53 protein. Some of these cells stabilize wild type (wt) protein while others may contain mutant protein. Furthermore, a mutation in one of the two alleles may not always lead to p53 protein accumulation as it is occasionally seen in tissues of Li-Fraumeni patients. As a result, the p53 positive stained cell does not necessarily contain a mutation in the p53 gene. The p53 gene is also activated by hypoxia, heat shock, exposure to nitric oxide and other stresses.

To identify individuals who are predisposed to elevated spontaneous mutations or who have had previous carcingen exposure, it is advantageous to use the smallest amount of PCR template that could result in an accurate picture of mutation load. HotStart PCR, which helps to prevent the formation of primer dimer, permits amplification of more dilute template. Using this technique, a 140 bp PCR segment was amplified from an extracted single DNA template using 60 cycles. Vogelstein, B., Kinzler, K. W., “Digital PCR”, Proc. Natl. Acad. Sci. U.S.A. 96:9236-9241 (1999). This technique involves the fewest number of PCR cycles required for dependable results reported to date.

Thus, there exists a need for a method to assess mutation load, both in subjects which do not yet exhibit signs of the disease, and subjects which are presently being treated using known cancer therapy to assess efficacy of treatment.

### SUMMARY OF THE INVENTION

In accordance with the present invention it has been discovered that the p53 gene is a useful reporter of somatic cell mutation analysis, including determination of mutation load. In one embodiment, the invention is a method for determining mutation load which involves identifying a somatic cell that contains accumulated levels of p53, amplifying DNA from the p53 gene in such cell and determining the frequency or nature of mutations in the amplified DNA. In another embodiment, the p53 gene is obtained from a single somatic cell.

In a further embodiment, the method of this invention involves a method for determining mutation load by identifying a somatic cell that contains accumulated levels of both p53 and altered levels of a protein which is the product of a gene whose expression is regulated by p53, amplifying DNA of the p53 gene in such cell, and determining the frequency or nature of mutations in the amplified DNA. Cells containing accumulated levels of p53 and altered levels of such other protein advantageously may be identified by immunohistochemical staining using antibodies to those proteins. Mutations may be identified by sequence analysis of the amplification product.

As described in greater detail below, the combination on microdissection of single cells with well preserved and intact genomic DNA and efficient amplification makes possible a mutation load assay from ethanol-fixed paraffin-embedded tissues. A robust method involves reproducible double immunohistochemical staining, efficient microdissection of single cells under conditions that preserve genomic DNA integrity, and efficient amplification of the p53 gene with low false negatives and low false positives. The method of this invention has been used successfully and analyze DNA templates as large as 1 kb, preferably as large as 2 kb. The method has been used to identify missense mutations in single cells from normal colon and other tissues. Specifically, mutations were identified in 50% of the amplified samples by sequence analysis of exons 5 to 9 of the p53 gene. In addition, rates of allele dropout and polymerase error, important to estimates of mutation load, were examined.

This invention is useful for assessing cancer risk and prognosis and monitoring the effectiveness of cancer therapy. The method also can be used to monitor the mutational status of individuals over extended periods of time, such as individuals who have environmental or inherited cancer risks. Changes in or accelerated progression of mutation load can signal increased risks for cancers or other cellular abnormalities.

### BRIEF DESCRIPTION OF THE DRAWINGS

FIG. 1, Panel A shows normal colon tissue with crypt cells stained positive for PCNA and for p53 (black arrow). FIG. 1, panel B shows normal lung tissue with bronchial epithelial cells. Cells are stained for PCNA (white arrow), and p53 (black arrow). FIG. 1, panel C shows benign proliferative lesion of the mammary gland. Cells are stained for p53 (white arrow) and for p53/PCNA (black arrow).

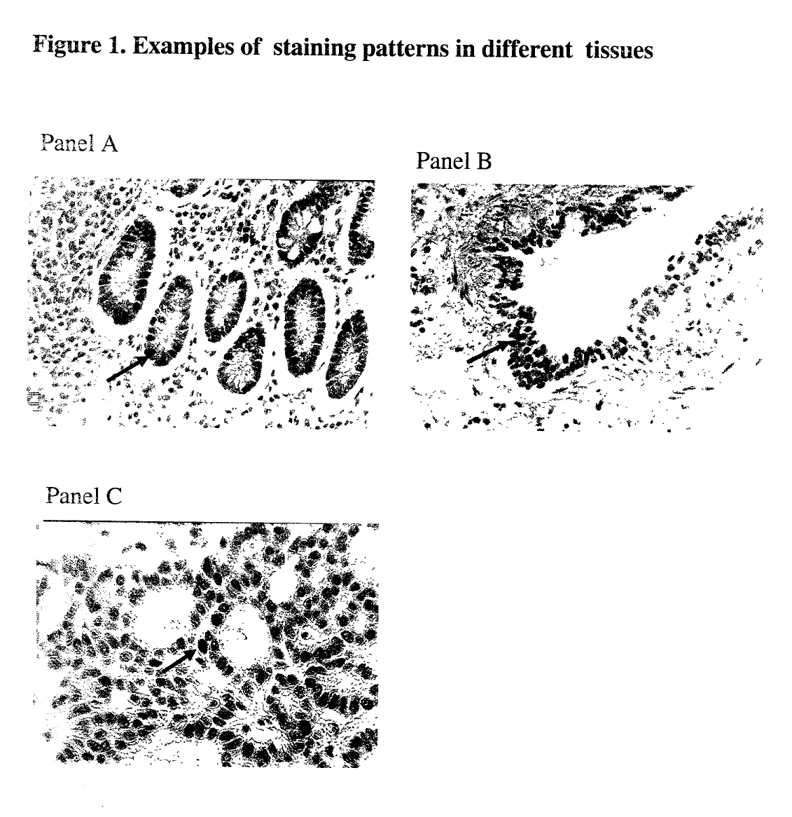

FIG. 2 shows single cell amplification by Stimulated-PCR, the technique discussed in Example II below. A 2 kb region of the p53 gene was amplified with 0.6 μl of p53(12983)30D (SEQ. ID. NO. 1) and p53(15036)33U (SEQ. ID. NO. 3) from genomic DNA of single microdissected cells for 40 cycles. Lanes 1 to 12 are single cell amplifications. Eleven out of 12 single sells were amplified. Lane 13=no DNA, Lane 14=positive control with 20 cells, M=DNA marker.

FIG. 3 shows examples of identified somatic mutations. Panels A through D are examples of identified somatic mutations in sequence analysis. Mutations of C-T, G-A, T-A and T-C were identified with second peak heights from 100% to 30% of the wild type. Causative mutations were found in 50%-70% of the amplified single cells. All mutations were confirmed by an additional sequencing in the opposite direction.

### DETAILED DESCRIPTION OF THE INVENTION

Somatic tissue for examination by the method of this invention may be obtained from any tissue source, e.g., colon, prostate, breast, skin, lung, etc. The tissue is prepared for identification of cells that have accumulated p53, as such cells are likely to have mutations in the p53 gene and other genes. Any method of preparing tissue samples which permits microdissection of cells and minimizes damage to nuclei may be used. A preferred method for tissue preparation involves ethanol fixation, paraffin embedment and EDTA steam heating to unmask antigenic sites. The method is described below in connection with a preferred embodiment.

Ethanol is a preferred tissue fixative, because it precipitates antigens and does not cause DNA crosslinking, as does formalin. Following ethanol fixation, tissue is embedded in paraffin and sliced in thin sections by standard procedures. Routine pathology sections conventionally are between 4 μm and 5 μm in diameter. When using this diameter, tissue sections consist not only of undamaged sections, but also contain some damaged nuclei, resulting in allele dropout. To enhance the possibility of obtaining undamaged nuclei and reducing the risk of dissecting damaged cell nuclei, large sections, e.g., about 6 μm or greater are preferred. Steam heating using an EDTA buffer has been found to yield reliable immunohistochemical staining and intact DNA. Taylor, C. R., Shi. S. R., Cote, R. J., “Antigen retrieval for immunohistochemistry status and need for greater standardization”, Applied Immunohistochemistry 4:144-146 (1996). Suitable conditions include a buffer containing 1 mM EDTA (pH 8.0) at 96 to 100° C. and heating for 5 minutes. Alternatively, steam heating can be performed using 20 mM HEPES/1 mM EDTA buffer (pH 8.1) with a pK much less affected by high temperature. Using these process enhancements, the size of the single stranded DNA has been determined to have an average length of 20 kb.

Cells that accumulate p53 or have altered levels of a protein which is the product of a gene whose expression is regulated by p53 may be identified by immunohistochemical staining. Monoclonal antibodies that may be used for this purpose are available commercially. See Examples, infra. Because mutant p53 accumulates in cells, staining for this protein is useful for identifying cells in which mutations have occurred. The p53 protein regulates the expression of a number of other genes, including PCNA, mdm2 and vEGF. Thus the levels of the proteins that are the products of these genes often are altered in cells containing mutant p53. For example, p53 down regulates PCNA, and mutatnt p53 may result in accumulated levels of PCNA in cells. The levels of other proteins under p53 control may be increased or decreased, depending on the mechanism of the control. The use of the levels of expression of one or more of such secondary proteins assists in diffrentiation between cells having enhanced levels of wt p53 resulting from natural physiological induction and the cells of interest having accumulated levels of mutant p53.

After preparation and staining of the paraffin-embedded tissue sections, single cells that stain positive for p53 and/or PCNA are microdissected from nontumorous tissues. Microdissection is performed by standard procedures.

Mutation analysis, including determination of mutation load, advantageously is determined by amplification and analysis of DNA from a single cell. The DNA is amplified by any procedure that efficiently reproduces DNA from the low template concentrations obtained from a single cell. A preferred amplification procedure, referred to herein as “Stimulated PCR,” has been found to yield sufficient DNA for sequence analysis using as fews as 40 cycles of amplification. This PCR process differs from known processes in that it substantially reduces the threshold effect of the template concentration on PCR efficiency. Additional advantages of Stimulated-PCR may include: inhibition of absorbance of the template to the tube surface; protection against minimal DNAase activity; addition of false priming sites for spurious extensions; activation by binding DNA polymerase; or direct stimulation of extension by DNA polymerase. Stimulated PCR is described in detail in the Examples, infra. In general, the technique is characterized by the use of a combination of a Taq polymerase High Fidelity and Taq DNA polymerase and by the incorporation of mouse genomic DNA having an average size of more than about 20 kb. The addition of mouse genomic DNA allows a wider range of annealing temperatures, a wider range of primer concentrations, less primer dimer formation, and higher product yield. Similar effects were observed by adding or supplementing bovine serum albumin (BSA), probably because BSA protein assists in keeping DNA polymerases in active forms.

Changes in the use of High Fidelity enzymes, commercially available and utilized by those of skill in the art, improve amplification yields. The highest yields are found when the High Fidelity enzymes are used in higher amounts than those typically used, e.g., at about 4-fold the amount recommended by the manufacturer (2.5 U Taq/GB-D DNA polymerases per 25 μl of reaction). Additionally, mixing 1 U of Platinum Taq with 1 U of the High Fidelity enzymes, which increases the unit ratio of Taq to GB-D by 2 fold, behaves better than the High Fidelity enzymes alone, indicating that not only the total units, but also the relative ratios of the enzymes are important. Another improvement that may be used to increase the fidelity of the amplification and to minimize primer-dimer formation is the incorporation of a Taq antibody to inactivate Taq DNA polymerase at room temperature. This improvement is used in so-called HotStart PCR. In the present method, it has been effective in preventing primer dimer formation using 40 to 45 cycles.

Primer design is an important factor in Stimulated PCR. Analysis of any region of the p53 gene may be used in the method of this invention. In a preferred embodiment, sequences from exons 5 to 9 of the p53 are amplified and analyzed, as a large majority of mutations occur in these regions. Table 1 presents a list of primers which have been utilized during development of this invention. The following primers have been found particularly effective for Stimulated-PCR:

| | |
|---|---|
| GCCGTCTTCCAGTTGCTTTATCTGTTCACT | (SEQ. ID. NO.1); |
| CCTGATGGCAAATGCCCCAATTGCAGGTAA | (SEQ. ID. NO.2); and |
| GTCAAGTAGCATCTGTATCAGGCAAAGTCATAG | (SEQ. ID. NO.3). Primer |
| TGTTCACTTGTGCCCTGACTTTCAACTCTG | (SEQ. ID. NO.4) was used in conjunction with |
| CCTGATGGCAAATGCCCCAATTGCAGGTAA | (SEQ. ID. NO.2) |

for half-nested PCR. The specific physical properties of these sequences are set forth in Table 1. Primer design was achieved using a strict criteria to ensure PCR specificity and efficiency, which is explained further in Example II below. For instance, it was discovered that primers with AA dinucleotides at the 3′ end formed fewer dimers.

TABLE 1
List of primers in the p53 gene

| Primer | | | | | PCR segment^{c} | | |
|---|---|---|---|---|---|---|---|
| | | Size | Tₘ | GC | Size | Tₘ | GC |
| Name^{a} | Sequence (5′-3′) | (base) | (^{o}C)^{b} | % | (bp) | (^{o}C)^{b} | % |
| p53(12983)30D | GCCGTCTTCCAGTTG | 30 | 64.3 | 46.7 | 1880 | 81.7 | 54.5 |
| | CTTTATCTGTTCACT | | | | | | |
| | [SEQ. ID NO. 1] | | | | | | |
| p53(14863)30U | CCTGATGGCAAATGC | 30 | 71.5 | 50 | | | |
| | CCCAATTGCAGGTAA | | | | | | |
| | [SEQ. ID NO. 2] | | | | | | |
| p53(12983)30D | GCCGTCTTCCAGTTG | 30 | 64.3 | 46.7 | 2053 | 81.2 | 53.1 |
| | CTTTATCTGTTCACT | | | | | | |
| | [SEQ. ID NO. 1] | | | | | | |
| p53(15036)33U | GTCAAGTAGCATCTG | 33 | 61.6 | 42.4 | | | |
| | TATCAGGCAAAGTCA | | | | | | |
| | TAG [SEQ. ID NO. 3] | | | | | | |
| p53(13005)30D | TGTTCACTTGTGCCC | 30 | 65 | 46.7 | 1858 | 81.7 | 54.5 |
| | TGACTTTCAACTCTG | | | | | | |
| | [SEQ. ID NO. 4] | | | | | | |
| p53(14863)30U | CCTGATGGCAAATGC | 30 | 71.5 | 50 | | | |
| | CCCAATTGCAGGTAA | | | | | | |
| | [SEQ. ID NO. 2] | | | | | | |

| | | | | | |
|---|---|---|---|---|---|
| p53(13016)24D | TGCCCTGACTTTCAA | 24 | 56.4 | 50 | D sequencing |
| | CTCTGTCTC | | | | |
| | [SEQ. ID NO. 5] | | | | |
| p53(13281)20D | AGGGTCCCCAGGCCT | 20 | 58.3 | 65 | D sequencing |
| | CTGAT | | | | |
| | [SEQ. ID NO. 6] | | | | |
| p53(13491)22U | GGCCACTGACAACCA | 22 | 57.5 | 54.5 | U sequencing |
| | CCCTTAA | | | | |
| | [SEQ. ID NO. 7] | | | | |
| p53(13950)20D | AGGTCTCCCCAAGGC | 20 | 59.5 | 65 | D sequencing |
| | GCACT | | | | |
| | [SEQ. ID NO. 8] | | | | |
| p53(14155)20U | GGGGCACAGCAGGCC | 20 | 61.7 | 70 | U sequencing |
| | AGTGT | | | | |
| | [SEQ. ID NO. 9] | | | | |
| p53(14506)19D | GGAGAGACCGGCGC | 19 | 58.4 | 68.4 | D sequencing |
| | ACAGA | | | | |
| | [SEQ. ID NO. 10] | | | | |
| p53(14487)24U | CGGCATTTGAGTGT | 24 | 57.1 | 45.8 | U sequencing |
| | TAGACTGGA | | | | |
| | [SEQ. ID NO. 11] | | | | |

a. The sequence of the p53 gene was from a revised version of X54156 in GenBank. As an example for p53 (12983)30D,p 53=thep53 gene, (12983)30D=5′ end of the primer begins at 12983 base position, and the length is 30 bases “downstream” (D) (i.e., in the direction of transcription). The precise sizes and locations of the PCR fragment can be obtained from the informative names.

b. Tₘ of the primer was estimated by the nearest neighbor method at 50 mM Kcl and 250 pM DNA and Tₘ of the PCR segment was estimated by the formula of Wetmur: Tₘ^{product}=81.5+6.6 log[K⁺=0.05 M]+0.41 (% G+% C)=675/length.

c. primer pairs of [SEQ. ID. NO. 1] and [SEQ. ID. NO. 2] and of [SEQ. ID. NO. 1] and [SEQ. ID. NO. 3] were used for Stimulated PCR; primer pair of [SEQ. ID. NO. 4] and [SEQ. ID. NO. 2] were for half-nested PCR, Primers [SEQ. ID. NO. 5] through [SEQ. ID. NO. 11] were for sequencing.

Because contamination is a significant consideration when only amplifying DNA product from a single cell, various sources of non-specific DNA and excessive PCR amplification must be eliminated. To decrease or eliminate these possible causes of contamination, the following steps were taken: i) negative controls without target DNA template are routinely performed with each assay, and ii) the preparation process was always performed in a special clean room. Moreover, if a number of microdissected single cells are found to contain different mutations, this is evidence that the finding is not attributable to a common source of contamination.

Once amplification is completed using the Stimulated-PCR process, the PCR product is sequenced according to techniques known in the art. Sequencing is described in Example III below. Primers that may be used for the sequencing process include:

| | |
|---|---|
| TGCCCTGACTTTCAACTCTGTCTC | (SEQ. ID. NO.5); |
| AGGGTCCCCAGGCCTCTGAT | (SEQ. ID. NO.6); |
| GGCCACTGACAACCACCCTTAA | (SEQ. ID. NO.7); |
| AGGTCTCCCCAAGGCGCACT | (SEQ. ID. NO.8); |
| GGGGCACAGCAGGCCAGTGT | (SEQ. ID. NO.9); |
| GGAGAGACCGGCGCACAGA | (SEQ. ID. NO.10); and |
| CGGCATTTTGAGTGTTAGACTGGA (SEQ. ID. NO.11). | |

Physical properties of these sequencing primers are set forth in Table 1. Table 3 shows the mutations identified using the assay method disclosed herein. Once the mutations are identified, to determine mutation load, an accurate estimate is made of both allele dropout and false positive mutations due to polymerase error. This process is detailed in Example IV.

Generally, allele dropout may be caused by failure to retrieve the entire cell nucleus due to microdissection, degradation or nicking of the genomic DNA, absorbance of the DNA to the tube surface, preferential amplification of one allele and/or preferential primer extension in the sequencing reaction. To eliminate some of these causes of allele dropout, the paraffin-embedding procedure has been altered, as detailed above, to increase the diameter of the section used. Allele dropout due to nicked template can be kept at a reasonably low level. Table 2 shows the size effect of single-stranded template on allele dropout for amplification of 2 kb. The staining process changes discussed above, as well as addition of mouse genomic DNA or BSA to the digestion reaction, further protect the target DNA from potential degradation and prevent absorbance to the tube surface. Preferential PCR amplification is diminished by primer design, reaction component changes and optimization of the thermocycling steps.

TABLE 2
List of Identified Mutations^{a}

| Single stranded template | | | Average size of single stranded template^{b} | | | | |
|---|---|---|---|---|---|---|---|
| No. | | No. | 3 kb | 4 kb | 6 kb | 10 kb | 20 kb |
| nicked | No. | allele | Allele | Allele | Allele | Allele | Allele |
| strands | combinations | dropout | dropout | dropout | dropout | dropout | dropout |
| 0 | 1 | 0/1 | 0% | 0% | 0% | 0% | 0.0% |
| 1 | 4 | 0/4 | 0% | 0% | 0% | 0% | 0.0% |
| 2 | 6 | 2/6 | 9.9% | 12.5% | 9.9% | 5.1% | 1.6% |
| 3 | 4 | 4/4 | 39.5% | 25.0% | 9.9% | 2.6% | 0.4% |
| 4¹ | 1 | 0/1 | No ampl | No ampl | No ampl | No ampl | No ampl |
| | | Total | 49.8% | 37.5% | 19.8% | 7.7% | 2.0% |

a. This estimate only considers the size effect of the single stranded template on allele dropout assuming 100% of amplification efficiency amplification. Allele dropout is defined as that the peak height of one allele is lower than 10% of the second allele at the heterozygote base position.

b. The average size of single stranded template can be determined in its denatured form on a agarose gel. If the size is 4 kb, the 2 kb PCR segment has 1/2 chance within an integrity template without nicking.

c. When all the four single stranded templates are nicked, there is only one combination of the four templates to be nicked and no amplification can occur.

Additionally, the error rate due to the use of two different polymerase systems may be calculated and used to adjust the final mutation load determination. Mutation analysis, including adjustment of the mutation load by expected allele dropout percentages and false positive mutations due to polymerase error, are shown in Example IV below.

The following examples further illustrate the invention. It is understood that modifications which do not substantially affect the various embodiments of this invention are also included within the invention as set forth in the claims. Accordingly, the following examples are not intended to limit the present invention.

### EXAMPLE I

### Immunohistochemical Staining and Microdissection

Fresh tissue from colon cancer patients was cut into 3-4 mm thin slices and immediately transferred into jars with the ethanol-based fixative (95% ethanol with 0.2 mM EDTA buffer, pH 8.0) for at least 12 hrs. The specimens were processed the following day, and paraffin embedded using standard procedures. From each tissue block, sections of 6 μm diameter were cut using a rotation microtome. The deparaffinization process included one xylene step at room temperature for 30 min with shaking every 5 min, followed by steps in alcohols of different concentrations. Steam heating at 96-100° C. for 5 minutes in 1 mM EDTA buffer (pH 8.0) was performed to unmask the antigenic sites.

The PCNA antibody (Ab-1 monoclonal mouse IgG antibody) (Oncogene Calbiocaem) was used in a concentration of 1:4000; the p53 antibody (mouse monoclonal antibody DO7) (Novocastra) was used in a concentration of 1:100. The tissue sections were double stained immunohistochemically for p53 and PCNA. Cells testing positive showed p53 positive nuclear staining (bright red), PCNA positive cytoplasmatic staining (light brown) or both PCNA and p53 positive staining (reddish brown).

The single cells were manually microdissected using an inverted microscope (Nikon TMS) and a mechanical micromanipulation system (Sutter Instruments). A tungsten needle was manipulated through a joystick. The microdissected cell was then picked up manually with a new 27 G ½″ needle, and transferred into a 0.2 mL PCR tube containing 5 μl digestion buffer: #3 High Fidelity buffer without Magnesium, 2 mg/ml Proteinase K (Qiagen), 3% Tween-20 detergent and 0.2 mM EDTA (pH 8.0). The single cell was digested at 50° C. for 16 hr and after the digestion, Proteinase K was inactivated at 90° C. for 10 minutes. This single cell was then amplified by Stimulated PCR as set forth below in Example II.

The DNA quality was estimated by examination of the size of the single stranded template. The remaining cells on the slide were scratched off, digested by Proteinase K and the genomic DNA was denatured and electrophoresed through a 1% standard agarose gel.

### EXAMPLE II

### Stimulated PCR

In order to detect mutations in the single cell chosen for microdissection isolation from the paraffin-embedded tissue, the single cell was subjected to the Stimulated PCR technique. In preparing for the Stimulated-PCR used to amplify the single cell mutations, primer selection is important. Here, all primers were designed and analyzed with Oligo 5 software (National Biosciences). T^{m} of the primer was estimated by the nearest neighbor method at 50 mM KCl and 250 pM DNA and Tm of the PCR segment was estimated by the formula of Wetmur: T^{m}_{product}=81.5+16.6 log[K⁺=0.05M]+0.41(% G+% C)−675/length. Wetmur, J. G., “DNA probes: Applications of the Principles of Nucleic Acid Hybridization”, Critical Rev. in Biochem and Mol Biol. 26:227-259 (199 1), the disclosure of which is incorporated herein by reference. The criteria for specificity included high-specificity with low base-pairing stability at the 3′ end, no primer-dimer or hairpin formation more than 3 bases at the 3′ end, no homo- or repeat-sequence at the 3′ end, and no false priming site more than 7 bases at the 3′ end for any strand and any segment. The primer also had no false priming site on the mouse p53 gene to generate spurious products. The primers used are shown in Table 1, above. For Stimulated PCR, primer GCCGTCTTCCAGTTGCTTTATCTGTTCACT (SEQ. ID. NO. 1) was used in conjunction with either

| | |
|---|---|
| CCTGATGGCAAATGCCCCAATTGCAGGTAA | (SEQ. ID. NO.2) |
| | and |
| GTCAAGTAGCATCTGTATCAGGCAAAGTCATAG | (SEQ. ID. NO.3). |

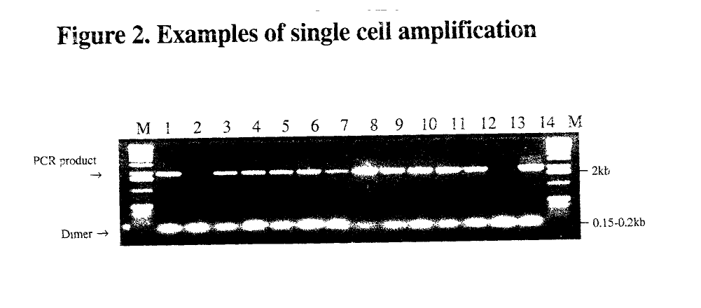

The results of a 2 kb region of the p53 gene amplification with 0.6 μl of p53(12983)30D (SEQ. ID. NO. 1) and p53(15036)33U (SEQ. ID. NO. 3) from genomic DNA of twelve single microdissected cells for 40 cycles are shown in FIG. 2.

The PCR mixtures contained a total volume of 25 μl: human genomic DNA from a dissected single cell; #3 Expanded High Fidelity buffer (Boehringer Mannheim); 3.5 mM MgCl₂; 500 μM of each dNTP; 2% DMSO; 0.2 to 0.6 μM of each of primers; a mixture of 1.25 U of Platinum Taq DNA polymerase High Fidelity (Taq/GB-D)/1.25 U of Platinum Taq DNA polymerase (GIBCO BRL); 5 μg of BSA and 25 ng of mouse genomic DNA with the average size more than 20 kb. The cycling conditions included denaturation at 92° C. for 12 seconds, annealing at 60° C. for 20 seconds, and elongation at 68° C. for 2 minutes for 40 or 45 cycles with a Perkin Elmer GeneAmp PCR system 9700. An additional 20 seconds of denaturation time proceeded the first cycle. Two to 4 μl of the PCR product was electrophoresed through a standard 1% agarose gel and then the gel was stained with ethidium bromide for UV photography by a CCD camera (Bio-Rad Gel Doc 1000) and Multi-Analyst software (version 1.1). Another nested or half nested PCR was performed for 12 to 15 cycles to obtain more product. For this additional half-nested PCR, primers

| | |
|---|---|
| CCTGATGGCAAATGCCCCAATTGCAGGTAA | (SEQ. ID. NO. 2) |
| and | |
| TGTTCACTTGTGCCCTGACTTTCAACTCTG | [SEQ. ID NO. 4] |

### EXAMPLE III

### Sequence Analysis

The PCR product was purified for two rounds using Microcon® 100 (Arnicon) to remove the unincorporated primer and primer dimers. Standard sequence analysis was performed using ABI 377 fluorescence sequencer and BigDye terminator chemistry with AmpliTaq FS DNA polymerase (PE Applied Biosystem). The primers used during the sequencing process were:

| | |
|---|---|
| TGCCCTGACTTTCAACTCTGTCTC | (SEQ. ID. NO.5); |
| AGGGTCCCCAGGCCTCTGAT | (SEQ. ID. NO.6); |
| GGCCACTGACAACCACCCTTAA | (SEQ. ID. NO.7); |
| AGGTCTCCCCAAGGCGCACT | (SEQ. ID. NO.8); |
| GGGGCACAGCAGGCCAGTGT | (SEQ. ID. NO.9); |
| GGAGAGACCGGCGCACAGA | (SEQ. ID. NO.10); and |
| CGGCATTTTGAGTGTTAGACTGGA | (SEQ. ID. NO.11). |

Any second peak was called if its height was more than 10% of the wild type. All mutations were confirmed by sequencing from the opposite direction.

### EXAMPLE IV

### Estimation of Mutation Load and Determination of Allele Dropout and Polymerase Error

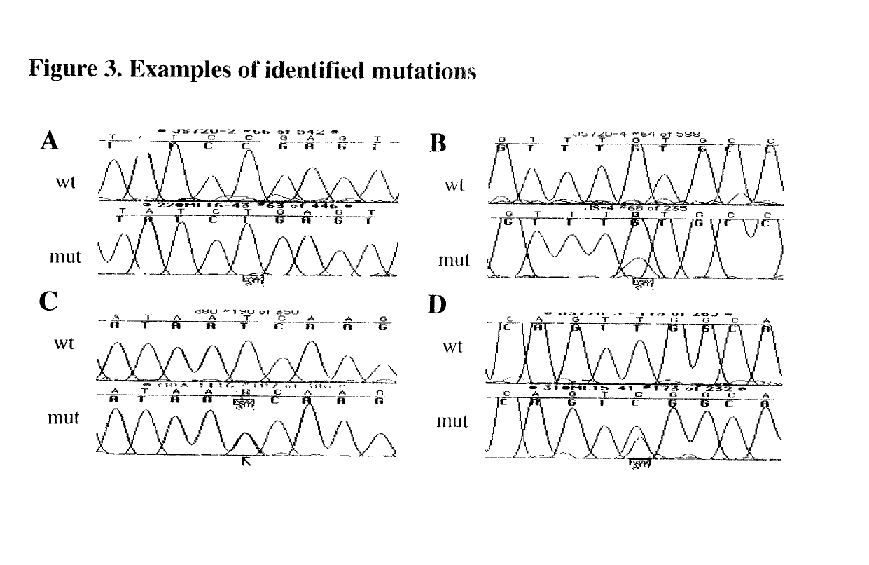

Using the technique detailed in Example II, a 2 kb segment in the human p53 gene spanning exons 5 to 9 was amplified with 60 to 80% success rate. Mutations were identified in 50% of amplified samples with p53 and/or PCNA positive staining, and are listed in Table 3. FIG. 3 shows examples of identified somatic mutations. Panels A through D are examples of identified somatic mutations in sequence analysis. Mutations G-A, T-A and T-C were identified with second peak heights from 100% to 30% of the wild type. Causative mutations were found in 50%-70% of the amplified single cells.

TABLE 3
List of Identified mutations^{a}

| | P53/PC | | | | | | | | |
|---|---|---|---|---|---|---|---|---|---|
| Mutation | N a | Mutation | Wt | Mt | Base | AA | AA | | Conser- |
| No. | Stain | Type | Base | Base | Position | Wt | Mt | Codon | vation |
| 1 | +/− | missense | A | G | 13074 | Lys | Arg | 132 | Y |
| 2 | +/− | missense | G | A | 13139 | Gly | Ser | 154 | Y |
| 3 | +/− | missense | A | G | 14015 | Thr | Ala | 230 | Y |
| 4 | +/− | missense | G | C | 14061 | Gly | Arg | 245 | Y |
| 5 | +/+ | missense | C | T | 13125 | Ser | Phe | 149 | N |
| 6 | +/− | missense | G | T | 13409 | Val | Leu | 217 | Y |
| 7 | +/− | missense | C | A | 14568 | Pro | His | 300 | N |
| 8 | +/− | missense | G | A | 13229 | Asp | Asn | 184 | N |
| 9 | +/− | missense | T | G | 14743 | Phe | Ser | 328 | Y |
| 10 | +/− | missense | G | A | 14574 | Gly | Glu | 302 | Y |
| 11 | +/− | missense | A | G | 13086 | Glu | Arg | 136 | N |
| 12 | +/− | missense | G | T | 14699 | Ser | lle | 313 | N |
| 13 | +/− | missense | T | G | 14743 | Phe | Ser | 328 | Y |
| 14 | +/− | Insertion | | A | 13607 | IVS 6 + 174 | | | |
| 15 | +/− | missense | G | A | 14574 | Gly | Glu | 302 | N |

a. All of the above were identified from 15 normal nontumorous colon cells with p53 and PCNA double straining. The missense mutations are either at a conservative site r are found in the p53 mutation database (http://www.iarc.fr/P53/index.html). Mutations were also identified in other normal cells of breast, lung, kidney and gallbladder.

Each allele has two single stranded templates in the heterozygous p53 genomic DNA in diploid cell. “Allele dropout” is defined herein as the situation in which the peak of one allele was less than 10% of the second peak at the heterozygous base position. The size effect of single stranded templates on allele dropout is summarized in Table 2 above. As shown, a low level of allele dropout due to nicked template can be achieved by maintaining 6 kb or more of single stranded template.

False positive mutations may result from a polymerase error in any of the first several PCR cycles. A false positive mutation is identified when a second peak is higher than 10% of the wild type peak in a wild type sample. The two enzyme system with Taq and GB-D DNA polymerases is estimated to have an error rate of 8.5×10⁻⁶ substitutions per base, this is expected to cause false positive mutation calls in 7% to 12% of the amplified samples or at 4×10⁻⁵ to 7×10⁻⁵ chance per nucleotide when amplified our to one single stranded template.

What is claimed is:

1. A method for determining mutation load which comprises identifying a somatic cell that contains accumulated levels of p53, amplifying DNA of the p53 gene from such cell and determining the frequency or nature of mutations in the amplified DNA.

2. The method of claim 1, in which the somatic cell that is identified also contains altered levels of a protein selected from the group consisting of PCNA and other proteins that are regulated by p53.

3. The method of claim 2, wherein the protein is PCNA.

4. The method of claim 2, wherein the protein is mdm2 or vEGF.

5. The method of claim 1, in which the somatic cell is identified by immunohistochemical staining for p53.

6. The method of claim 2, in which the somatic cell is identified by immunohistochemical staining for p53 and a protein selected from the group consisting of PCNA and other proteins that are regulated by p53.

7. The method of claim 1 or 2, in which the amplification is conducted in the presence of mouse DNA or bovine serum albumin or both.

8. The method of claim 1 or 2, wherein the DNA that is amplified is from exons 5 to 9 of the p53 gene.

9. The method of claim 1 or 2, wherein the DNA that is amplified is at least 1 kb in size.

10. The method of claim 1 or 2, wherein the DNA that is amplified is at least 2 kb in size.

11. The method of claim 1 or 2, in which the amplification is conducted in the presence of mouse DNA having an average size of at least about 20 kb.

12. The method of claim 1 or 2, in which the method is performed on a single somatic cell which is obtained by microdissection from a paraffin-embedded tissue section.

13. The method of claim 12, in which the tissue section is fixed with ethanol.

14. The method of claim 12 in which the tissue section is subjected to steam heating in the presence of EDTA to facilitate unmasking of antigen sites.

15. The method of claim 1 or 2, in which the amplification step utilizes two different DNA polymerases.

16. The method of claim 15, in which the two DNA polymerases are Platinum Taq DNA polymerase High Fidelity (Taq/GB-D) and Platinum Taq DNA polymerase.

17. The method of claim 11, in which amplification comprises use of primers of the sequence

| | |
|---|---|
| GCCGTCTTCCAGTTGCTTTATCTGTTCACT with either | (SEQ. ID. NO. 1) |
| CCTGATGGCAAATGCCCCAATTGCAGGTAA or | (SEQ. ID. NO. 2) |
| GTCAAGTAGCATCTGTATCAGGCAAAGTCATAG. | (SEQ. ID. NO. 3) |

18. The method of claim 17, which further comprises use of primers of the sequence

| | |
|---|---|
| GCCGTCTTCCAGTTGCTTTATCTGTTCACT | (SEQ. ID. NO. 1) |
| and | |
| CCTGATGGCAAATGCCCCAATTGCAGGTAA. | (SEQ. ID. NO. 2) |

19. The method of claim 17, which further comprises use of primers of the sequence

| | |
|---|---|
| GCCGTCTTCCAGTTGCTTTATCTGTTCACT | (SEQ. ID. NO.1) and |
| GTCAAGTAGCATCTGTATCAGGCAAAGTCATAG | (SEQ. ID. NO.3). |

20. The method of claim 12, in which the paraffin-embedded tissue section is prepared from a sample that originated from a patient that is at risk for developing a cancerous condition.

21. The method of claim 12, in which the paraffin-embedded tissue section is prepared from a sample that originated from a patient that is currently receiving treatment for a present cancer condition.

22. The method of claim 21, in the treatment is radiation treatment.

23. The method of claim 21, in the treatment is cytotoxic drug treatment.

24. The method of claim 21, in the treatment is gene therapy treatment.

25. The method of claim 1 or 2, in which the frequency or nature of mutations is determined by sequence analysis which utilizes one or more of

| | |
|---|---|
| TGCCCTGACTTTCAACTCTGTCTC, | (SEQ. ID. NO. 5) |
| AGGGTCCCCAGGCCTCTGAT, | (SEQ. ID. NO. 6) |
| GGCCACTGACAACCACCCTTAA, | (SEQ. ID. NO. 7) |
| AGGTCTCCCCAAGGCGCACT, | (SEQ. ID. NO. 8) |
| GGGGCACAGCAGGCCAGTGT, | (SEQ. ID. NO. 9) |
| GGAGAGACCGGCGCACAGA, or | (SEQ. ID. NO. 10) |
| CGGCATTTTGAGTGTTAGACTGGA | (SEQ. ID. NO. 11) |
| as primers. | |
